(19) United States
(12) Patent Application Publication
(10) Pub. No.: US 2021/0085215 A1
(43) Pub. Date: Mar. 25, 2021

(54) ECG-BASED CARDIAC WALL THICKNESS ESTIMATION

(71) Applicant: Biosense Webster (Israel) Ltd., Yokneam (IL)

(72) Inventors: Shmuel Auerbach, Kerem Maharal (IL); Liat Tsoref, Tel Aviv (IL); Matityahu Amit, Cohav-Yair Zur-Yigal (IL); Yariv Avraham Amos, Tzorit (IL); Avi Shalgi, Zichron Ya'acov (IL)

(21) Appl. No.: 17/016,611

(22) Filed: Sep. 10, 2020

Related U.S. Application Data
(60) Provisional application No. 62/903,851, filed on Sep. 22, 2019.

(51) Int. Cl. A61B 5/107; A61B 5/042; A61B 5/00; G06N 3/08; G06N 3/04

(52) U.S. Cl. CPC A61B 5/1075; A61B 5/042; G06N 3/0454; G06N 3/088; A61B 5/7267

(57) ABSTRACT

A system includes an interface and a processor. The interface is configured to receive a plurality of electrophysiological (EP) measurements performed in a heart of a patient. The processor is configured to estimate a wall thickness at a specified location of the heart based on the EP measurements.

### CROSS-REFERENCE TO RELATED APPLICATIONS

This application claims the benefit of U.S. Provisional Patent Application 62/903,851, filed Sep. 22, 2019, whose disclosure is incorporated herein by reference.

### FIELD OF THE INVENTION

The present invention relates generally to processing of electrophysiological signals and ablation, and specifically to estimating properties of cardiac wall tissue using machine learning (ML).

### BACKGROUND OF THE INVENTION

A number of methods may be used to estimate a thickness of a cardiac wall, such as ultrasound, fluoroscopy, and MRI imaging. The estimated wall thickness may be further correlated with electrophysical signals to estimate an injury of the cardiac wall tissue. For example, Takeshi Sasaki et al. describe in “Myocardial Structural Associations with Local Electrograms: A Study of Post-Infarct Ventricular Tachycardia Pathophysiology and Magnetic Resonance Based Non-Invasive Mapping,” Circulation Arrhythmia and Electrophysiology, December, 2012; 5(6): 1081-1090, significant associations between left ventricular wall thickness, post infarct scar thickness, and intramural scar location seen in MRI, and local endocardial electrogram bipolar/unipolar voltage, duration, and deflections on electroanatomical mapping.

### SUMMARY OF THE INVENTION

An embodiment of the present invention that is described herein after provides a system including an interface and a processor. The interface is configured to receive a plurality of electrophysiological (EP) measurements performed in a heart of a patient. The processor is configured to estimate a wall thickness at a specified location of the heart based on the EP measurements.

In some embodiments, one or more of the EP measurements include intra-cardiac electrograms (EGMs).

In some embodiments, the EP measurements further include respective locations in the heart at which the EGMs were acquired.

In an embodiment, one or more of the EP measurements include body surface electrocardiograms (ECGs).

In some embodiments, the processor is configured to estimate the wall thickness using a model defined over the EP measurements, and to refine the model based on results of an ablation procedure applied at the specified location of the heart.

In another embodiment, the model is a trained machine learning (ML) model. In yet another embodiment, the ML model comprises at least one type of autoencoder comprising an encoder coupled to a decoder.

In an embodiment, the at least one autoencoder includes a first autoencoder configured to operate on the EGMs and a second autoencoder configured to operate on the ECGs.

In some embodiments, the results of the ablation procedure include one or more of (i) a temperature rise associated with the ablation procedure, and (ii) a change in tissue impedance associated with the ablation procedure.

There is additionally provided, in accordance with another embodiment of the present invention, a method including receiving a plurality of electrophysiological (EP) measurements performed in a heart of a patient. A wall thickness is estimated at a specified location of the heart based on the EP measurements.

### BRIEF DESCRIPTION OF THE DRAWINGS

The present invention will be more fully understood from the following detailed description of the embodiments thereof, taken together with the drawings in which:

FIG. 1 is a schematic, pictorial illustration of a catheter-based electrophysiological (EP) sensing, signal-analysis, and IRE ablation system, according to an exemplary embodiment of the present invention;

FIG. 2 is a flow chart of training and use for inference, of a machine learning model to estimate cardiac wall thickness, according to an exemplary embodiment of the present invention;

FIG. 3 illustrates a deep learning algorithm for wall thickness estimation based on autoencoders and a fully connected layer, according to an exemplary embodiment of the present invention; and

FIG. 4 is a schematic illustration of autoencoder architecture used in the deep learning algorithm of FIG. 3, according to an exemplary embodiment of the present invention.

### DETAILED DESCRIPTION OF EMBODIMENTS

### Overview

Cardiac ablation is a common procedure that is used to treat arrhythmias by forming lesions in cardiac tissue of a patient. Such lesions may be formed by irreversible electroporation (IRE), or other types of ablative energy, such as radiofrequency (RF), both of which can be applied using a catheter. In IRE ablation, the catheter is maneuvered such that electrodes disposed on a distal end of the catheter are in contact with, or in close proximity to, the tissue. Then high voltage bipolar pulses are applied between the electrodes, and strong electric field pulses produced in tissue cause cell death and lesion production. In RF ablation, an alternating RF current is applied to the tissue by one or more electrodes, causing cell death by heat.

In to order be effective, a tissue ablation has to be transmural, i.e., to penetrate into the depth of the tissue. However, “over-ablation” may create undesired damage to tissue (including, rarely, perforation of the cardiac wall) or harm to adjacent structures such as the esophagus which may be behind the cardiac tissue. Hence, it is important to be able to assess cardiac wall thickness (e.g. atrial or ventricular wall) just before and/or during ablation, in order to use optimal ablation parameters during the procedure.

Different imaging modalities may be employed to assess cardiac wall thickness, including magnetic resonance imaging (MRI), computerized tomography (CT), ultrasound, and more. However, using these modalities adds to the cost and the complexity of the procedure. Moreover, the spatial resolution of these modalities may be on the order of the tissue thickness, which may yield less accurate estimation of the actual wall thickness during ablation.

Embodiments of the present invention that are described hereinafter provide systems and methods to estimate the thickness of a cardiac wall, i.e., an atrial wall or a ventricular wall, just before and/or during ablation with limited up to date information at hand. In some embodiments, a machine learning (ML) model, such as an artificial neural network (ANN), is provided to allow for this estimation using only EP data, such as multi-channel (e.g., 12-lead) body-surface electrocardiogram (ECG), and intra-cardiac electrograms (EGM) acquired by electrodes of a catheter just before or during the ablation procedure.

In some embodiments, the ML model is trained using data comprising EP data (multi-channel ECGs and EGMs—the later also known as intra-cardiac ECG (IcECG)), patient medical history, and 3D location information of the data collected. The model is optimized to gain, via the training, predictive power of the wall thickness using ground truth data, such as atrial/ventricular wall thickness assessed by imaging modalities such as ultrasound, CT, MRI or similar imaging modalities.

The training data may also include (e.g., incorporate), along with the above data items, data collected after the start of ablation, and further initial ablation data, such as a temperature rise profile and/or an impedance change during ablation. (The temperature rise profile is detectable very quickly, typically 10 mSec or 100 mSec, in an ablation that typically takes between four (4) to sixty (60) seconds).

Just before and/or during a new ablation procedure (i.e., during inference), the model uses, as noted above, only EP data, including ECG and EGMS and subsequent ablation data (i.e., any subsequent data acquired during the ablation procedure) for a specific patient, in further assessing wall thickness of the patient.

While the ANN model is used here as an example, a person skilled in the art may choose from among other ML models available for use, such as decision tree learning, support vector machines (SVM), and Bayesian networks. ANN models include, for example, convolutional NN (CNN), autoencoder, and probabilistic neural network (PNN). Typically, the one or more processors used (collectively named hereinafter “processor”) are programmed in software containing a particular algorithm that enables the processor to conduct each of the processor-related steps and functions outlined above. Typically, the training is done using a computing system comprising multiple processors, such as graphics processing units (GPU) or tensor processing units (TPU). However, any of these processors may also be central processing units (CPUs).

The ability to assess cardiac wall thickness just before ablation, as well as during ablation, i.e., in real time, based on at least part of the data referred to above using an ML algorithm, allows a simple assessment of cardiac wall thickness, and may lead to a more accurate ablation time, and typically to an improvement in outcome of the ablation procedure as well.

### System Description

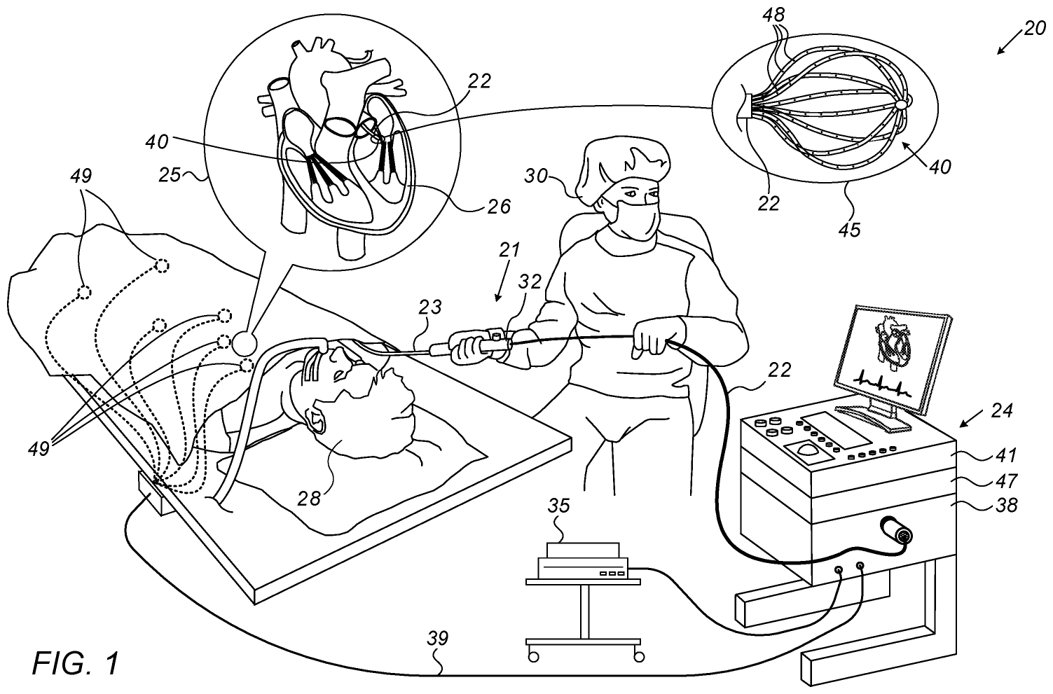

FIG. 1 is a schematic, pictorial illustration of a catheter-based electrophysiological (EP) sensing, signal-analysis, and IRE ablation system 20, according to an exemplary embodiment of the present invention. System 20 may be, for example, a CARTO® 3 system, produced by Biosense-Webster. As seen, system 20 comprises a catheter 21, having a shaft 22 that is navigated by a physician 30 into a heart 26 (inset 25) of a patient 28. In the pictured example, physician 30 inserts shaft 22 through a sheath 23, while manipulating shaft 22 using a manipulator 32 near the proximal end of the catheter.

In the embodiment described herein, catheter 21 may be used for any suitable diagnostic purpose and/or tissue ablation, such as electrophysiological mapping of heart 26 and IRE ablation, respectively. An ECG recording instrument 35 may receive various types of ECG signals sensed by system 20 during the process.

As shown in inset 25, a distal end of shaft 22 of catheter 21 is fitted with a multi-electrode basket catheter 40. Inset 45 shows an arrangement of multiple electrodes 48 of basket catheter 40. The proximal end of catheter 21 is connected to a control console 24, to transmit, for example, electrograms acquired by electrodes 48.

Console 24 comprises a processor 41, typically a general-purpose computer, with suitable front end and interface circuits 38 for receiving EP signals (e.g., ECGs and EGMS) as well as non-EP signals (such as position signals) from electrodes 48 of catheter 21. For this purpose, processor 41 is connected to electrodes 48 via wires running within shaft 22. Interface circuits 38 are further configured to receive ECG signals, such as from a 12-lead ECG apparatus that can be ECG recording instrument 35, as well as non-ECG signals from surface body electrodes 49. Typically, electrodes 49 are attached to the skin around the chest and legs of patient 28. Processor 41 is connected to electrodes 49 by wires running through a cable 39 to receive signals from electrodes 49.

Four of surface body electrodes 49 are named according to standard ECG protocols: RA (right arm), LA (left arm), RL (right leg), and LL (left leg). A Wilson Central Terminal (WCT) may be formed by three of the four named body surface electrodes 49, and a resulting ECG signal, V_{WCT}, is received by interface circuits 38.

During an EP mapping procedure, the locations of electrodes 48 are tracked while they are inside heart 26 of the patient. Such tracking may be performed using the Active Current Location (ACL) system, made by Biosense-Webster, which is described in U.S. Pat. No. 8,456,182, whose disclosure is incorporated herein by reference.

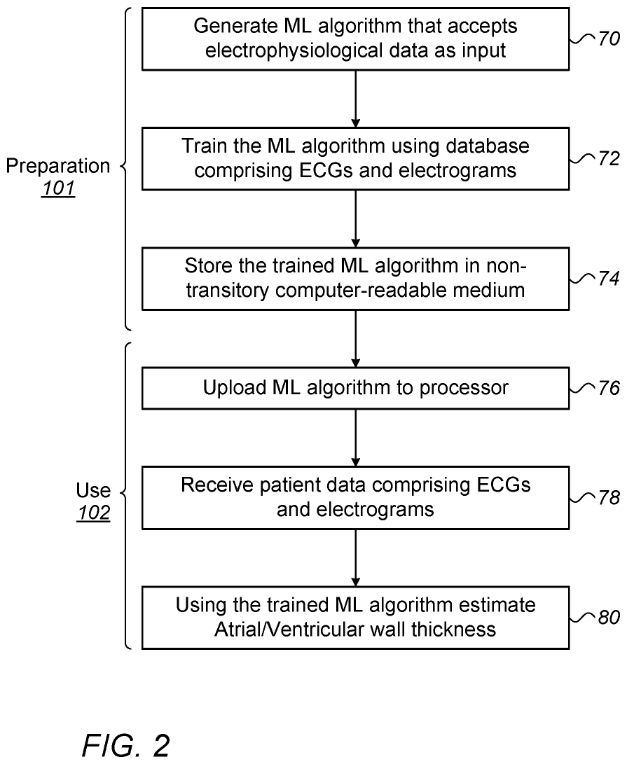

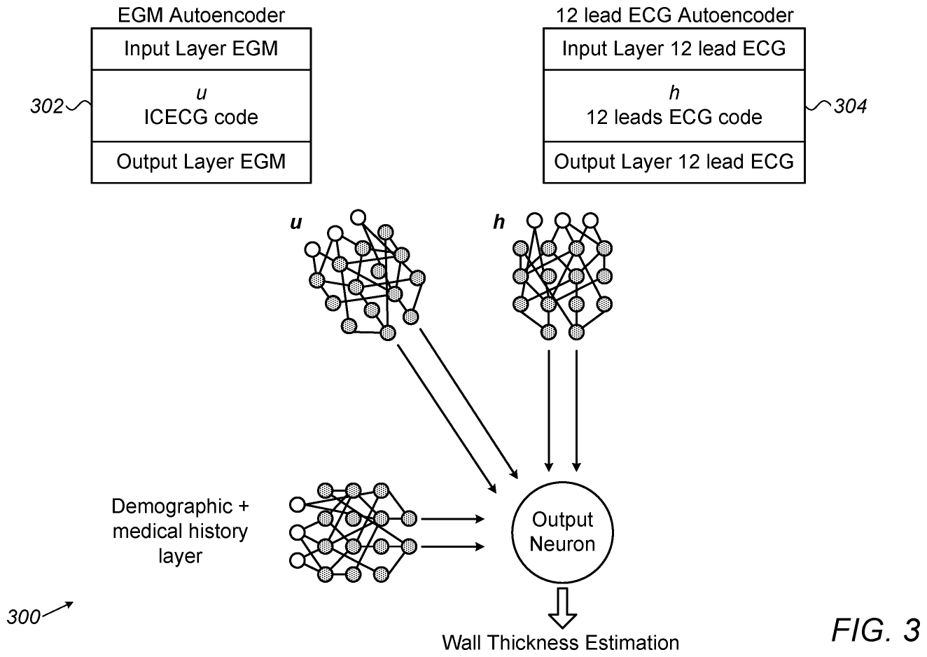

The processor may thus associate any given signal received from electrodes 48, such as EGMs, with the location at which the signal was acquired. Processor 41 uses information contained in these signals to construct an EP map, such as a local activation time (LAT) map, to present on a display. In the shown embodiment, using an algorithm comprising an ML algorithm applied to EP and other data, as described in FIGS. 2 and 3, processor 41 estimates cardiac wall thickness.

To perform IRE ablation, electrodes 48 are connected (e.g., switched) to an IRE pulse generator 47 comprising a processor-controlled switching circuitry (e.g., an array of relays, not shown) in console 24. Using the wall-thickness information, processor 41, or the physician, may select which electrodes to connect to pulse-generator 47 to apply IRE pulses (via the switching circuitry).

During RF ablation, initial and subsequent ablation data comprise at least one of the following: an IRE energy profile, a temperature rise, and a change of impedance. They may be used in further assessing (e.g., in real time) wall thickness of the patient, as described in FIG. 2.

Processor 41 is typically programmed in software to carry out the functions described herein. The software may be downloaded to the processor in electronic form, over a network, for example, or it may, alternatively or additionally, be provided and/or stored on non-transitory tangible media, such as magnetic, optical, or electronic memory. In particular, processor 41 runs a dedicated algorithm as disclosed herein, included in FIG. 3, that enables processor 41 to perform the disclosed steps, as further described below.

ECG-Based Cardiac Wall Thickness Estimation Using ML

FIG. 2 is a flow chart of training, and use for inference, of a machine learning model to estimate cardiac wall thickness, according to an exemplary embodiment of the present invention.

The algorithm, according to the presented exemplary embodiment, is divided into two parts: algorithm preparation 101 and algorithm use 102.

Algorithm preparation carries out a process that begins at ML modeling step 70, to generate an ML algorithm for estimating cardiac wall thickness.

Next, using a database including ECGs and EGMs, a processor trains the algorithm (e.g. the ANN and preprocessing parts), at an ML algorithm training step 72. In step 72, the processor uses training data, including ground truth data, to train the ML model. Training data is formed from:

1. Multi-channel (e.g., 12-lead) ECG data

2. Electrograms with 3D information of the cardiac tissue collection location

3. Anatomical location of each intracardiac electrode—Based on another ML model/3D segmentation of the atria using MFAM).

4. Diagnostic catheter details

5. Patient demographic information, (for example, gender, age, height, weight)

6. Patient medical history

Ground truth data is formed from:

7. Atrial/ventricular wall thickness assessed by imaging modalities such as ultrasound, CT, MRI or similar imaging modalities.

Additional training data may also include an ablation transmitting energy profile, as well as ablation related parameters such as a temperature rise and/or an impedance change and/or an elasticity change and/or a stiffness change during ablation. The data items #1-#6 above, while collected after ablation begins, and/or the additional collected training data, are collectively termed herein “ablation data.”

The algorithm preparation ends with storing the trained model in a non-transitory computer-readable medium, such as a disc on key (memory stick), at a trained model storing step 74. In alternative embodiments, the model is sent in advance, and its optimized parameters (such as weights of an ANN) are sent separately after training.

Algorithm use 102 carries out a process that begins at algorithm uploading step 76, during which a user uploads either an entire ML model or its optimized parameters (e.g., weights) to a processor. Next, the processor, such as processor 28, receives patient inference data, for example, the aforementioned ECGs and EGMs from electrodes 49 and 48, respectively, at patient data receiving step 78.

Next, using the trained ML model for inference, the processor inputs data from a selected patient to the trained model, and implements an algorithm on the model so that the model is able to output an atrial or a ventricular wall thickness of the patient only from limited data available, such as the aforementioned EP data, at cardiac wall thickness estimation step 80. After being installed on the processor, the trained model may be used with multiple patients.

In some embodiments, the NN model outputs a statistical distribution of thicknesses, and a peak of the distribution may be selected in a subsequent step, i.e., beyond those included in the NN model, to determine the most likely wall thickness value.

The example flow chart shown in FIG. 2 is chosen purely for the sake of conceptual clarity. The present embodiment may also comprise additional steps of the algorithm, such as receiving indications of the degree of physical contact of the electrodes with diagnosed tissue. This and other possible steps are omitted from the disclosure herein purposely in order to provide a more simplified flow chart.

### ML Algorithm Description

FIG. 3 illustrates a deep learning algorithm 300 for wall thickness estimation based on autoencoders and a fully connected layer, according to an exemplary embodiment of the present invention. The method comprises providing a deep learning supervised framework for the estimation, and uses electrograms, 12-lead ECG, anatomical data, catheter details, demographic data of a patient, and the medical history of the patient, (corresponding to training data items #1-#6 above). The method may also use a temperature rise and/or an impedance change during ablation.

In the method, two autoencoders 302 and 304 (described in more detail below) are applied to perform dimensionality reduction to a set of features from the 12-lead ECG and/or from the intracardiac ECG. The method uses a fully connected layer based on those features and based on medical history information including, but not limited to, a NYHA (New York Heart Association) score, a CHA2DS2-VASc score, and an AF duration, as well as demographic data (e.g. age, gender, height and weight). A regression analysis is then performed in order to estimate the thickness of the cardiac wall.

As stated above, the method uses two autoencoders 302 and 304. An autoencoder comprises two parts: an encoder and a decoder. The encoder maps an input (in FIG. 3, an ECG signal and/or an EGM signal) to a hidden representation (h or u, respectively) via a nonlinear transformation. The decoder then maps the hidden representation back to reconstructed data via another nonlinear transformation. Equations 1 and 2 represent the mappings:

h=f(ECG,θ_{encoder}), ECG′=g(h,θ_{decoder}), Eq. 1

u=f(EGM,Ø_{encoder}), EGM′=g(u,Ø_{decoder}), Eq. 2

Where θ_{encoder}, θ_{decoder} are weights for the ECG signal reconstruction, and Ø_{encoder}, Ø_{decoder} are weights for the EGM signal reconstruction.

The same network architecture is used for ECG and EGM reconstruction, so that the nonlinear functions f and g are substantially the same. Using a minimize L2 normalization function between a set of autoencoders provides a set of θ_{encoder}, θ_{decoder} weights for ECG signal reconstruction and a set of Ø_{encoder}, Ø_{decoder} weights for EGM reconstruction.

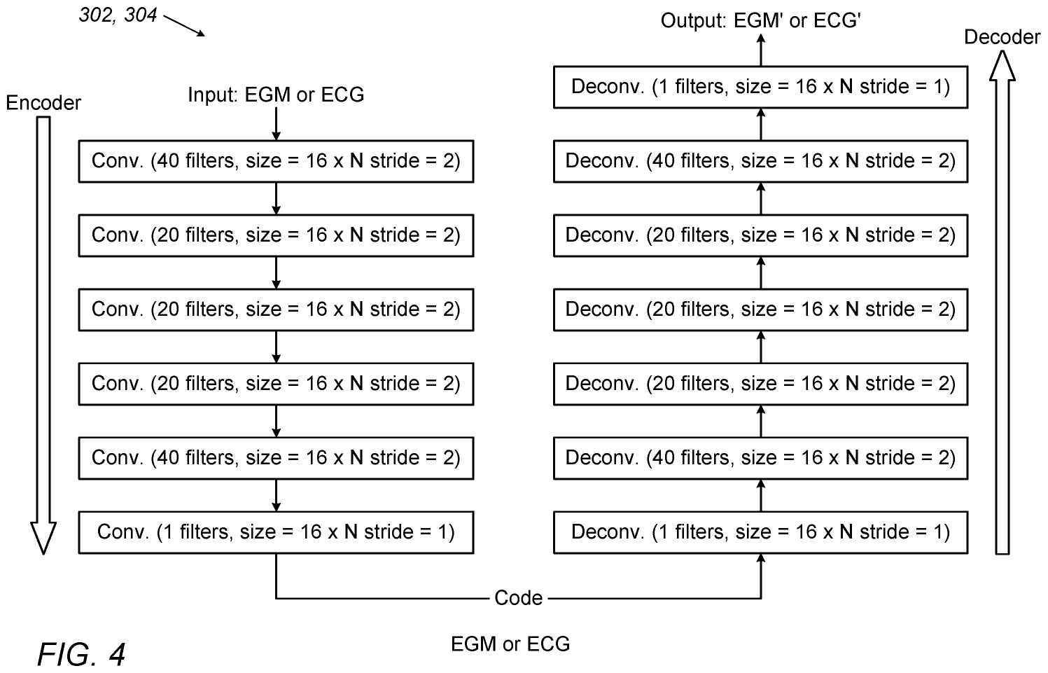

FIG. 4 is a schematic illustration of autoencoder architecture used in the deep learning algorithm of FIG. 3, according to an exemplary embodiment of the present invention. In particular, the autoencoder architecture is used for compressing and learning feature space of electrograms and/or 12-lead ECG. Each autoencoder is implemented using a fully connected convolutional neural network (FCN) of an encoder and a decoder with a predefined number of layers, as shown in the figure. In the encoder, the size of EGM/ECG signals is reduced, and the signals are encoded into low dimensional features. The decoder attempts to reconstruct an output depending on the low dimensional features. Embodiments of the invention employ rectifier linear units (ReLUs) as activation functions for hidden layers. There is no activation function for the output layer in the FCN model. In addition, each hidden layer undergoes batch normalization.

The encoder contains a series of layers, where each individual layer is composed of a convolutional layer, a batch normalization layer, and an activation layer. The input layer is defined by the original signals with a size of 1024×N, where N represents the number of inputted channels. Thus N=12 for a 12-lead ECG, and for intracardiac ECG signals N corresponds to the number of electrodes on the catheter-acquiring signals. For example, N=20 is used for a PentaRay or Lasso catheter, and N=64 used for the basket catheter of FIG. 1. It will be understood that the catheters referred to hereinabove are examples, and that the scope of the present invention comprises any cardiac catheter.

A convolutional process with 40 filters of size 16×N and a stride of 2 is applied on the first layer. The next three convolutional layers all have 20 filters of size 16×N with a stride of 2. Then, the next layer consists of 40 filters of size 16×N with a stride of 2. The last layer has one filter of size 16×1 with a stride of 1. The down-sampling process is achieved using a stride of 2. Through the encoding process, a 32×N dimensional feature map is obtained. This feature map also represents the compressed data and is 32 times smaller than the original data size.

The decoder part of the autoencoder is inversely symmetric to the encoder. Here, the deconvolutional layers proceed to up-sample the feature maps so as to recover structural details. As for the output layer, a final deconvolutional layer with one filter of size 16×N and a stride of 1 produces the output signal.

Returning to FIG. 3, hidden representations h and u, patient medical history information (NYHA score, CHA2DS2-VASc score, AF duration and persistent AF duration) and patient demographic data (age, gender, height and weight) serve as a feature space (light gray circles in FIG. 3) for a fully connected neural network with four hidden layers (dark gray circles). In some embodiments, the feature space also comprises at least one of a temperature rise and an impedance change inputs.

The outputs from the hidden layers are then inserted into an output neuron which estimates the wall thickness of the heart.

The entire network is trained using a backpropagation algorithm that attempts to minimize an L2 regularization function, shown in equation 3.

J(φ)=Σᵢ∥WTᵢ−WTᵢ′∥²−β∥φ∥, Eq. 3

where J(φ) is a loss function, WTᵢ represents an atrial/ventricular wall thickness taken from ultrasound or CT, MRI, or similar imaging modalities of a subject i, WTᵢ′ is the cardiac wall thickness estimated based on the suggested method, φ is a weight of a fully connected layer, and β is a regularization parameter. In a disclosed embodiment β is set at 0.01.

The backpropagation algorithm is performed to minimize the loss function J(φ).

In exemplary embodiments of the invention a deep learning regressor for cardiac wall thickness is obtained after learning the optimal values (in an L2 regularization sense) of the set of parameters φ, h, u, θ_{encoder}, θ_{decoder}, Ø_{encoder}, Ø_{decoder}.

The disclosed embodiment provides, by way of example only, specific numbers, such as number of filters. In general, such numbers may be modified. While the description above refers to an ablation procedure, and to measuring a tissue wall thickness for the procedure, it will be appreciated that the description may be adapted, mutatis mutandis, to measuring tissue wall thickness absent an ablation procedure. Thus, the scope of the present invention comprises cardiac procedures with or without an ablation procedure.

It will thus be appreciated that operating the algorithm described above enables the processor to approximate the thickness of a cardiac wall. The value may be incorporated into a GUI of an ablation system. Alternatively, or additionally, the thickness value may be presented as a number on a “wall” drawing, or the wall thickness may be displayed graphically according to a scale of the heart image presented on an ablation system display.

It will consequently be appreciated that the embodiments described above are cited by way of example, and that the present invention is not limited to what has been particularly shown and described hereinabove. Although the embodiments described herein mainly address cardiac diagnostic applications, the methods and systems described herein can also be used in other cardiac medical applications that require estimation of cardiac wall thickness.

It will be therefore further appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system for estimating properties of cardiac wall tissue, the system comprising:
an interface, configured to receive a plurality of electrophysiological (EP) measurements performed in a heart of a patient; and
a processor, configured to estimate a wall thickness at a specified location of the heart based on the EP measurements.

2. The system according to claim 1, wherein one or more of the EP measurements comprise intra-cardiac electrograms (EGMs).

3. The system according to claim 2, wherein the EP measurements further comprise respective locations in the heart at which the EGMs were acquired.

4. The system according to claim 1, wherein one or more of the EP measurements comprise body surface electrocardiograms (ECGs).

5. The system according to claim 1, wherein the processor is configured to estimate the wall thickness using a model defined over the EP measurements, and to refine the model based on results of an ablation procedure applied at the specified location of the heart.

6. The system according to claim 5, wherein the model is a trained machine learning (ML) model.

7. The system according to claim 6, wherein the ML model comprises at least one type of autoencoder comprising an encoder coupled to a decoder.

8. The system according to claim 7, wherein the at least one autoencoder comprises a first autoencoder configured to operate on the EGMs and a second autoencoder configured to operate on the ECGs.

9. The system according to claim 5, wherein the results of the ablation procedure comprise one or more of (i) a temperature rise associated with the ablation procedure, and (ii) a change in tissue impedance associated with the ablation procedure.

10. A method for estimating properties of cardiac wall tissue, the method comprising:
receiving a plurality of electrophysiological (EP) measurements performed in a heart of a patient; and
estimating a wall thickness at a specified location of the heart based on the EP measurements.

11. The method according to claim 10, wherein one or more of the EP measurements comprise intra-cardiac electrograms (EGMs).

12. The method according to claim 11, wherein the EP measurements further comprise respective locations in the heart at which the EGMs were acquired.

13. The method according to claim 10, wherein one or more of the EP measurements comprise body-surface electrocardiograms (ECGs).

14. The method according to claim 10, wherein estimating the wall thickness comprises using a model defined over the EP measurements, and refining the model based on results of an ablation procedure applied at the specified location of the heart.

15. The method according to claim 14, wherein the model is a trained machine learning (ML) model.

16. The method according to claim 15, wherein the ML model comprises at least one type of autoencoder comprising an encoder coupled to a decoder.

17. The method according to claim 16, wherein the at least one autoencoder comprises a first autoencoder configured to operate on the EGMs and a second autoencoder configured to operate on the ECGs.

18. The method according to claim 14, wherein the results of the ablation procedure comprise one or more of (i) a temperature rise associated with the ablation procedure, and (ii) a change in tissue impedance associated with the ablation procedure.
